(19) United States
(12) Patent Application Publication
(10) Pub. No.: US 2007/0123801 A1
(43) Pub. Date: May 31, 2007

(54) Wearable, programmable automated blood testing system

(76) Inventors: Daniel Goldberger, Boulder, CO (US); Eric Shreve, Louisville, CO (US); Wayne Siebrecht, Golden, CO (US); Benny Pesach, Rosh Haayin (IL); Gidi Pesach, Kfar Vitkin (IL); Gabby Bitton, Jerusalem (IL); Ron Nagar, Tel Aviv (IL); Dalia Argaman, Hod-Hasharon (IL); Stephen Bellomo, Zircon Yacov (IL); Robert Larson, Perkasie, PA (US)

Correspondence Address: PATENTMETRIX, 14252 CULVER DR. BOX 914, IRVINE, CA, 92604, US

(21) Appl. No.: 11/287,897

(22) Filed: Nov. 28, 2005

(51) Int. Cl. A61B 5/00; C12Q 1/54; A61B 5/05; A61B 5/02

(52) U.S. Cl. 600/583; 600/490; 600/584; 600/347; 600/365; 435/14; 600/483

(57) ABSTRACT

The present invention is a programmable, automated device for the measurement and analysis of blood analytes and blood parameters. The device components are combined in a single apparatus and either programmed to initiate automatic, periodic blood sampling or initiate automatic blood sampling via operator input. The device operates automatically to draw blood samples at suitable, programmable frequencies to analyze the drawn blood samples and obtain the desired blood readings.

### FIELD OF THE INVENTION

The present invention relates generally to a device and method for monitoring blood parameters and blood constituents, and in particular, to a device and system for portable and programmable periodic measurement of blood glucose and other analytes.

### BACKGROUND OF THE INVENTION

Patient blood chemistry and devices, systems and methods of monitoring patient blood chemistry are important diagnostic tools in patient care. Measuring blood analytes and parameters often yields much needed patient information, allowing for drug administration to be carried out in the proper amounts and time periods. Blood analytes and parameters tend to change frequently, however, especially in the case of a patient under continual treatment, thus making the measurement process tedious, frequent, and difficult to manage.

Diabetes mellitus, for example, can contribute to serious health problems because of the physical complications that can arise from abnormal blood glucose levels. In the United States alone, it is estimated that over 11 million people suffer from diabetes. The two most common forms of diabetes are Type I, juvenile-onset, and Type II, adult-onset. Type I diabetes destroys the vast majority of the insulin-producing beta cells in the pancreas, thus forcing its sufferers to take multiple daily insulin injections. Type II diabetes is usually less severe than Type I, causing a decreased level of endogenous insulin production in the body, and can often be controlled by diet alone.

The body requires insulin for many metabolic processes; it is chiefly important for the metabolism of glucose. If normal blood glucose levels are maintained throughout the day, it is believed that many of the physical complications associated with diabetes could be avoided. Maintaining a consistent and normal blood glucose level is a challenging and arduous task as the diabetic's blood glucose level is prone to wide fluctuations, especially around mealtime. Many diabetics are insulin dependent and require routine and frequent injections to maintain proper blood glucose levels.

Controlling glucose levels requires continuous or frequent measurements of blood glucose concentration in order to determine the proper amount and frequency of insulin injections. The ability to accurately measure analytes in the blood, particularly glucose, is important in the management of diseases such as diabetes. Blood glucose levels must be maintained within a narrow range (about 3.5-6.5 mM). Glucose levels lower than this range (hypoglycemia) may lead to mental confusion, coma, or death. High glucose levels (hyperglycemia) cause excessive thirst and frequent urination. Sustained hyperglycemia has been linked to several severe complications of diabetes, including kidney damage, neural damage, and blindness.

Prior art systems have conventionally focused upon manually obtaining blood samples from capillary blood test devices for intermittent use. Such electronic devices are generally handheld and require several manual operations. For example, conventional glucose measurement techniques typically require assembling a clean lancet into a spring-loaded lancing device, triggering the lancing device to puncture a convenient part of the body (normally a fingertip) with a lancet, milking the finger to produce a drop of blood at the impalement site, and depositing the drop of blood on a measurement system (such as an analysis strip to be read via an electronic meter). This lancing method, at typical measurement frequencies of two to four times a day, is both painful and messy for the patient. In addition, the patient must dispose of the blood contaminated material, where proper disposal may be inconvenient. The pain and inconvenience has additional and more serious implications of noncompliance. Patients generally avoid maintaining the recommended regimen of blood glucose measurement and thereby run the risk of improper glucose levels and consequent harmful effects.

SureStep® Technology, developed by Lifescan, is one example of a conventional Point-of-Care home monitoring system. The SureStep® Technology, in its basic form allows for simple, single button testing, quick results, blood sample confirmation, and test memory. In operation, the SureStep® Point-of-Care home monitoring system employs three critical steps to obtain a measurement. In a first step, the blood sample is applied to the test strip. In a second step, the glucose reacts with the reagents in the test strip. The intensity of color formed at the end of the reaction is proportional to the glucose present in the sample. In a third step, the blood glucose concentration is measured with SureStep® meters. Reflectance photometry quantifies the intensity of the colored product generated by the enzymatic reaction. The system is calibrated to yield plasma glucose values.

U.S. Pat. No. 6,192,891, assigned to Beckton, Dickson, and Company, discloses “in a diagnostic and medication delivery system, a unit comprising: a housing, said housing having a first compartment adapted to removably receive and store a medication delivery pen and a second compartment adapted to removably receive and store a lancer; and a monitor integrated in the housing for monitoring a characteristic of a sample of a bodily fluid, wherein said monitor is not integrally attached to said medication delivery pen, such that a user is provided with the flexibility to use different medication delivery pens with said system but only one monitor.”

U.S. Pat. No. 6,849,237, assigned to Polymer Technology Systems, Inc., discloses “a diagnostic apparatus for testing body fluids, comprising: a base having: a slot adapted for receipt of a first test strip; a first display configured to display the concentration of an analyte in a body fluid sample contained in the first test strip; and a docking station adapted to detachably receive a portable tester; and a portable tester detachably mountable to said base, said portable tester having a second display and a port adapted to receive a second test strip containing a body fluid sample, said portable tester operable to test the sample contained in said second test strip when detached from said base.”

The conventional point-of-care and home monitoring glucose meters described above, however, have substantial disadvantages. Since such portable meters can be used by a patient without a practitioner or supervisor, numerous errors can arise from these unsupervised procedures that may result in serious health risks for patients, which knowingly, or inadvertently, are not in compliance with medical directives. Additionally, patients often forget, or in some instances forego, conducting and correctly recording their glucose levels as measured by the instrument.

In the light of above described disadvantages, there is a need for programmable, automated systems and methods that can provide comprehensive, accurate, and easy-to-use blood parameter testing. More specifically, what is needed is a programmable, automated system and method for obtaining blood samples at predetermined time intervals for convenient testing of blood parameters and also for data management of measurement results, thus avoiding human recording errors.

What is also needed is a programmable and portable, automated system and method for obtaining blood samples at predetermined time intervals for convenient testing of blood parameters.

What is also needed is a programmable and wearable, automated system and method for obtaining blood samples at predetermined time intervals for convenient testing of blood parameters.

### SUMMARY OF THE INVENTION

The present invention is a programmable, automated device for measurement and analysis of blood analytes and blood parameters. The device components are preferably combined in a single apparatus and either programmed to initiate automatic, periodic blood sampling or initiate automatic blood sampling via operator input. The device operates automatically to draw blood samples at suitable, programmable frequencies to analyze the drawn blood samples and obtain the desired blood readings.

In one embodiment, the automated blood testing device comprises a sampling and measurement unit for obtaining a blood sample and measuring blood analytes and blood parameters in said sample, wherein the sampling and measurement unit further comprises a lancet, a lancet launching mechanism, and a blood analyte measuring element; and a control unit for controlling the periodic sampling of blood and measurement of blood analytes and blood parameters, wherein the control unit is programmable to initiate blood sampling for measurement of blood analytes at pre-determined time intervals. Preferably, the control unit is a microprocessor or state machine.

The device is preferably portable and wearable. In a wearable configuration, the automated device further comprises an inflatable cuff, which is used for obtaining a blood sample via applying pressure. The inflatable cuff may also be used for non-invasive measurement of blood pressure.

The lancet is preferably a single-use lancet. The blood contacting elements are disposable and contained in a disposable cartridge or cassette. The blood sampling and measurement unit further comprises at least one measurement element for measuring at least one blood parameter. In one embodiment, the measurement element is a glucose oxidase test strip and further embodies a sensor. The sensor is contained in a sensor cassette that is disposable. The sensor cassette may be coded or keyed to insure proper operation. The sensor cassette preferably comprises at least one pre-calibrated single use sensor. In another embodiment, the sensor cassette comprises a plurality of sensors arranged in a multiple layer tape structure, wherein each single-use sensor is advanced sequentially and positioned for direct contact with a blood sample through an advancement means. Optionally, the sensor cassette includes a plurality of sensor cassettes, each comprising a different type of sensor.

In one embodiment, the sensor is an electrochemical sensor capable of detecting the presence of and enabling the measurement of the level of an analyte in a blood sample via electrochemical oxidation and reduction reactions at the sensor. In another embodiment, the sensor is an optochemical sensor capable of detecting the presence of and enabling the measurement of the level of an analyte in a blood or plasma. In another embodiment, the sensor determines the oxygenation level of the blood and uses the oxygenation level to calibrate the glucose calculation. In addition, the sensor determines the hemoglobin concentration and/or hematocrit of the blood and calibrates the glucose calculation.

The present invention also discloses a method for automatically measuring blood analytes and blood parameters, the method comprising: programming a control unit to obtain a blood sample from a blood sampling and measurement unit at predetermined time intervals; initiating a blood sample from the blood sampling and measurement unit at said predetermined time interval; launching a lancet via an automated launching mechanism located in said blood sampling and measurement unit when said blood sample is initiated; allowing said lancet to pierce the skin; retracting said lancet after said blood sample is obtained; and measuring analytes and parameters of said blood sample using an analyte measuring element in said blood sampling and measurement unit.

The aforementioned and other embodiments of the present invention shall be described in greater depth in the drawings and detailed description provided below.

### BRIEF DESCRIPTION OF THE DRAWINGS

These and other features and advantages of the present invention will be appreciated, as they become better understood by reference to the following Detailed Description when considered in connection with the accompanying drawings, wherein:

FIG. 1 is a block diagram illustrating the major components of an embodiment of the programmable, automated blood parameter testing apparatus of the present invention;

FIG. 2 is a block diagram of one embodiment of a sampling and measurement unit of the programmable, automated blood parameter testing apparatus of the present invention;

FIGS. 3a-3d illustrate a sensor tape as a multiple-layer element, as used in one embodiment of the present invention;

FIG. 4 is an illustration of a sensor cassette as used in the automated blood analysis automated system of the present invention;

FIGS. 5a and 5b are schematic diagrams of embodiments of wearable, programmable, automated blood parameter testing apparatus of the present invention;

FIG. 6 is a schematic diagram of one embodiment of the automated blood parameter testing apparatus of the present invention, incorporating a pressure cuff; and

FIG. 7 illustrates one use of a monitor in conjunction with the programmable, automated blood parameter testing apparatus of the present invention.

### DETAILED DESCRIPTION OF THE INVENTION

The present invention is directed towards a programmable, automated device for measurement and analysis of blood analytes and blood parameters. The device components are combined in a single apparatus and either programmed to initiate automatic, periodic blood sampling or initiate automatic blood sampling via operator input. The system operates automatically to draw blood samples at suitable, programmable frequencies to analyze the drawn blood samples and obtain the desired blood readings such as glucose levels, hematocrit levels, hemoglobin blood oxygen saturation, blood gasses, lactates or any other parameter as would be evident to persons of ordinary skill in the art.

The present invention is also directed towards a programmable, automated blood parameter testing device that includes a reusable sensor or a plurality of single use sensors that are packaged together in a cassette (hereinafter, referred to as “sensor cassette”) for obtaining blood measurements. The sensors are preferably electrochemical or optochemical sensors, but other options such as sensors that support optical blood measurements (without relying on chemical reactions between the sample of blood and a chemical agent embedded in the sensor) are disclosed. The present invention also discloses apparatuses and methods that employ components of manual test systems (e.g. blood glucose test strips) for use in an automated measurement system.

The present invention is also directed towards programmable, automated devices for measurement and analysis of blood analytes and blood parameters that are wearable. In one embodiment, the present invention is a programmable, automated blood parameter testing device that is advantageously integrated with a conventional blood pressure cuff or bladder. The inflatable bladder may optionally be employed for squeezing blood from the measurement site and also enables measurement of blood pressure non-invasively, in addition to the capillary blood parameter.

The present invention is also directed towards an integrated, automated blood parameter measurement and analysis system that employs a method of data transmission between the automated measuring system and portable monitors.

In addition, the present invention is directed towards features of the automated blood analysis and measurement system, such as, but not limited to storage of measurement results for trending or later download and alerts or alarms based on predefined levels or ranges for blood parameters.

As referred to herein, the terms “blood analyte(s)” and “blood parameter(s)” refers to such measurements as, but not limited to, glucose level; ketone level; hemoglobin level; hematocrit level; lactate level; electrolyte level (Na⁺, K⁺, Cl⁻, Mg²⁺, Ca²⁺); blood gases (pO₂, pCO₂, pH); blood pressure; cholesterol; bilirubin level; and various other parameters that can be measured from blood or plasma samples.

In one embodiment, the integrated, automated blood parameter analysis and measurement system comprises an automated blood parameter testing apparatus for measuring blood glucose levels.

Reference will now be made in detail to specific embodiments of the invention. While the invention will be described in conjunction with specific embodiments, it is not intended to limit the invention to one embodiment. Thus, the present invention is not intended to be limited to the embodiments described, but is to be accorded the broadest scope consistent with the disclosure set forth herein.

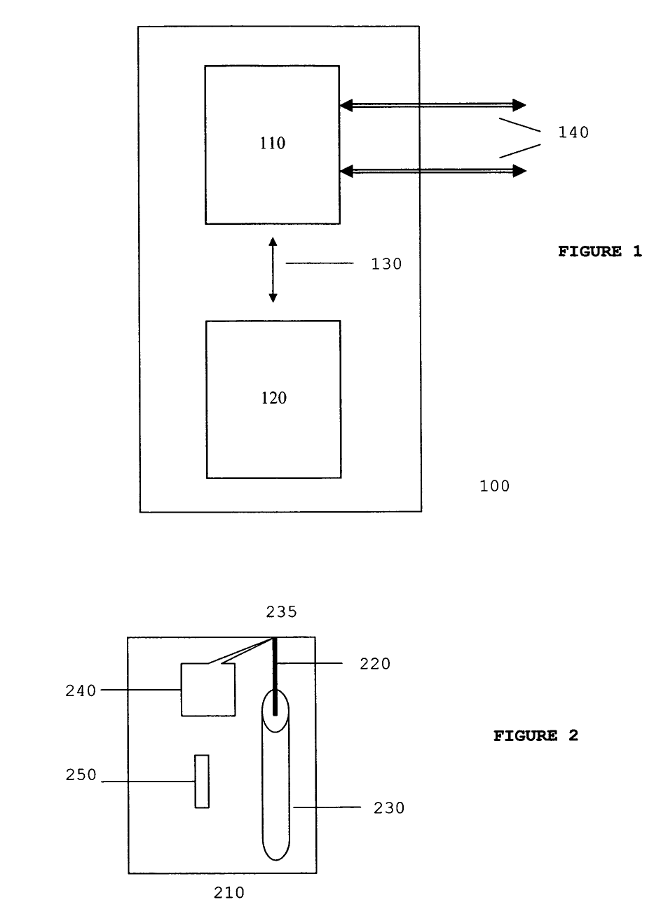

FIG. 1 is a block diagram illustrating the major components of an embodiment of the programmable, automated blood parameter testing apparatus of the present invention. Referring to FIG. 1, automated blood testing device 100 comprises a programmable control unit 110 for controlling the automatic operation of the system and a sampling and measurement unit 120 for obtaining the blood sample and measuring the analytes. The programmable control unit 110 enables automated blood sampling and analysis at predetermined intervals or time periods. In addition, the programmable control unit 110 can optionally be programmed to initiate blood sampling and measurement based upon a 24-hour time clock. Thus, the patient's blood sampling can be scheduled to record measurements throughout the day, at the same time each day, or can be changed according to an individual daily schedule. It is preferred that a measurement is scheduled for predetermined time periods which include, but are not limited to, one-, two-, and four-hour time periods.

For example, but not limited to such example, an operator or patient can program the unit to automatically measure blood analytes via initiation of a blood sampling and measurement unit 120 every four hours. It is also possible to program measurements at longer or shorter predetermined intervals. In addition, the operator or patient can initiate on demand testing. Programmable control unit 110 enables the display of test results as soon as thirty seconds after the blood sample reaches the measuring element.

In one embodiment, control unit 110 comprises a general purpose programmable microprocessor unit (not shown), as are well known to persons of ordinary skill in the art. In an alternate embodiment, control unit 110 comprises a state machine implemented in software and at least one processor. The programmable control unit 110 communicates with sampling and measurement unit 120 via an internal communication link 130. Internal communication link 130 may either be wired or wireless and may be based on a digital data link or on analog signals. Besides controlling and synchronizing functions for proper automated operation of the automated blood testing device 100, control unit 110 also includes required alert and built-in test capabilities. For example, but not limited to such example, the programmable control unit includes alert features to detect cuff inflation and lancet position for accurately obtaining a blood sample. Programmable control unit 110 also enables the user to define a reference range or reference values for the blood parameters measured by automated blood testing device 100. Thus, if a measurement is above or below the defined range or values, control unit 110 issues an alarm.

Programmable control unit 110 is also preferably equipped with external communication links 140 that may optionally include interfaces to external automated systems such as, but not limited to, portable monitors, printers, hospital data network(s), external processors and display units, and other monitoring automated systems. The connection between the control unit and the various possible external units can be made via any of the known wired or wireless communication methods, as are well-known in the art.

FIG. 2 is a block diagram of one embodiment of a sampling and measurement unit of the programmable, automated blood parameter testing device of the present invention. In one embodiment, blood sampling and blood analyte measurement means 200 are assembled in a disposable cartridge 210. Disposable cartridge 210 preferably comprises a lancet 220, for piercing skin to obtain a blood sample. Lancet 220 is housed in an automated launching mechanism 230 that launches the lancet 220 when an indication is made that a blood sample needs to be obtained, allows the lancet 220 to pierce the skin, and retracts the lancet 220 after the blood sample is obtained. The automated launching mechanism 230 may be mechanical (such as spring or cam driven) or electrical (such as electromagnetically or electronically driven). In one embodiment, automated launching mechanism 230 is a spring-loaded launching mechanism. Lancet 220 is completely shielded by the cartridge 210 when it is not in position for lancing.

Disposable cartridge 210 may contain a single lancet 220 for single patient use or optionally, a plurality of lancets, wherein the lancet is replaced for each measurement. Further, the system may be programmed to pierce the same spot on the skin for every measurement or to target a different spot with each measurement. Adjacent spots may be 2 mm or more apart.

At the point where the lancet pierces the skin, disposable cassette 210 also contains a narrow opening 235 leading to reservoir 240. Narrow opening 235 causes capillary forces that allow the blood sample to be channeled into reservoir 240. From reservoir 240, the blood sample is carried through small passages (not shown), to the blood analyte measuring element 250 contained within cartridge 210. In an alternative embodiment, blood analyte measuring element 250 may be integrated with lancet 220.

Referring back to FIG. 2, in one embodiment blood analyte measuring element 250 is a glucose oxidase test strip, preferably disposable, as are well-known to those of ordinary skill in the art. In another embodiment, blood analyte measuring element 250 is a sensor for performing blood analyte measurements. A single pre-calibrated and reusable sensor may be employed. In another embodiment, a plurality of single use sensors may be employed. Each single-use sensor is advanced sequentially and positioned for direct contact with a blood sample through an advancement means.

In one embodiment, the sensor is preferably an electrochemical sensor capable of detecting the presence of and enabling the measurement of the level of an analyte in a blood sample via electrochemical oxidation and reduction reactions at the sensor. Optionally, the sensor is an optochemical sensor capable of detecting the presence of and enabling the measurement of the level of an analyte in a blood or plasma sample via optochemical oxidation and reduction reactions at the sensor.

In another embodiment the sensor may optionally be a surface or miniature container, such as but not limited to a capillary tube, enabling storage of the blood sample for optical measurements. In this embodiment, both a light source and a light detector are used for measuring the blood analyte based on reflected, transmitted or other known optical effects such as Raman Spectroscopy, NIR or IR Spectroscopy, FTIR or fluoroscopy.

When multiple single-use sensors are used, one of the various methods available for packaging multiple sensors may be employed. Packaging options preferably include, but are not limited to: embedding a plurality of sensors in a multi-layered tape structure encapsulated in a compact cassette formation; attaching a plurality of sensors to a tape; or packaging a plurality of sensors in a drum that enables singular selection of a sensor.

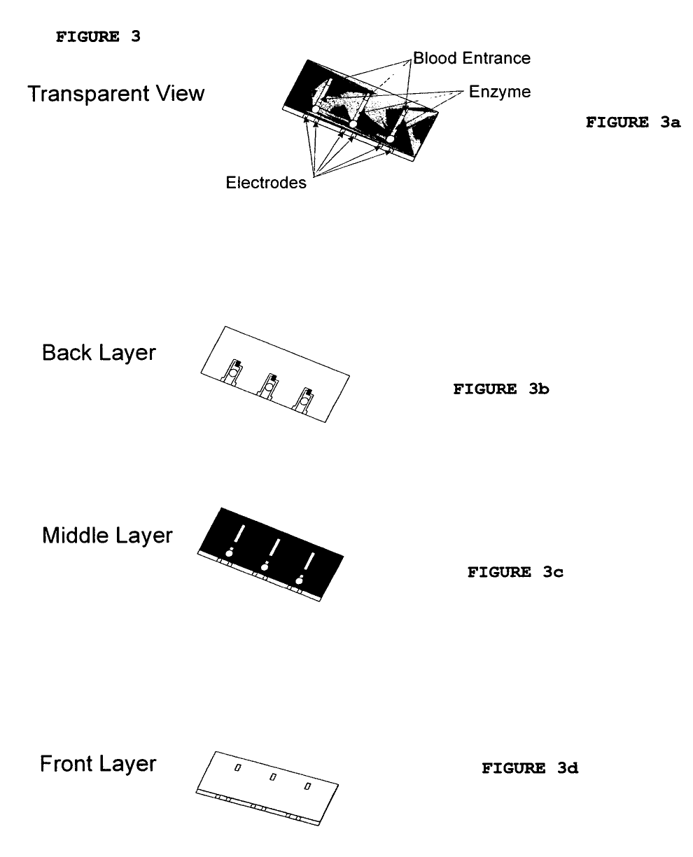

FIGS. 3a-3d illustrate a sensor tape as a multiple-layer element, as used in one embodiment of the present invention. FIG. 3a illustrates a transparent view of the multi-layer sensor tape as used in a preferred embodiment of the present invention, and described in further detail below. FIG. 3b depicts the back layer of the sensor tape; FIG. 3c illustrates the middle layer of the sensor tape; and FIG. 3d illustrates the front layer of the sensor tape as used in one embodiment of the present invention. Sensor tape preferably comprises at least one sensor, and even more preferably comprises a plurality of sensors.

In one arrangement of sensor tape comprises a front layer (shown in FIG. 3d), a middle layer (shown in FIG. 3c), substantially coplanar with the front layer, that is capable of transporting a blood sample by means of at least one capillary channel and further includes a suitable enzyme coating; and a back layer (shown in FIG. 3b), underlying the middle transporting layer, that comprises a plurality of electrochemical sensor electrodes for sensing required blood analytes such as, but not limited to glucose. Positioned at one end of the at least one capillary channel in the middle transport layer is a hole provided for an air outlet.

The front layer of sensor tape, and thus each sensor, may optionally be coated with a membrane for blocking the enzyme layer. When using a membrane coating to block the enzyme layer, sensor measures the plasma analyte level, such as plasma glucose level instead of the blood analyte level.

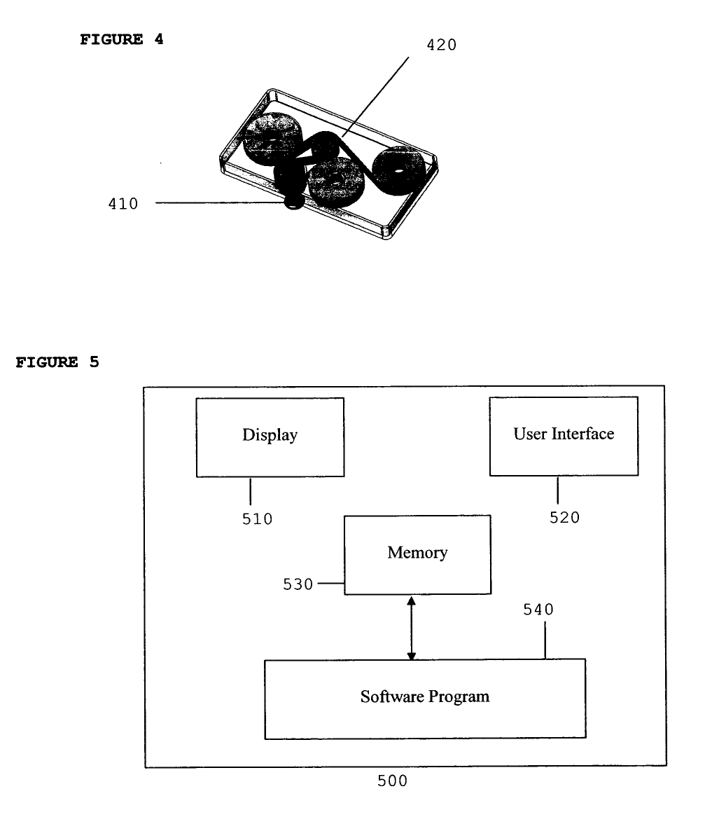

FIG. 4 is an illustration of a sensor cassette as used in the automated blood analysis automated system of the present invention. Single use sensors are preferably packaged into a sensor cassette that is replaced periodically. Such cassettes are known in the art and one such cassette is shown in FIG. 4. In one embodiment, the sensor cassette is assembled as a part of the cartridge containing lancets and the entire assembly is disposable.

The sensor cassette consists of an advancement mechanism comprising a cylindrical element 410 that rotates the sensor tape 420 to bring a sensor in contact with the blood sample. Thus, between measurements, the system moves a new sensor forward, thereby replacing the one used in the previous measurement. In one design, sensor cassette also stores the consumed test supplies and sample waster. An external waste container (not shown) may optionally be used to store the waste fluid and/or consumed test supplies.

In addition, sensor cassette may optionally include different types of single use sensors in one cassette, wherein each sensor is capable of measuring a different type of blood analytes or blood parameters. In this case, sensor selection is made based upon either operator programming or selection before usage. In another optional embodiment, sensor cassette may include a plurality of cassettes, each comprising a different type of sensor. The same automated blood sampling means is used for each measurement.

The use of single-use sensors (similar to the use of finger stick sensors) eliminates the need for time-consuming operator-directed automated system calibration procedures. In particular, each sensor cassette can be factory pre-calibrated.

In another embodiment, the disposable elements are mechanically, electrically, or otherwise keyed to mate with the reusable elements. Mechanical keys can take the form of a variety of three-dimensional, mating shapes, including, but not limited to cylinders, squares, or polygons of various configurations. Electrical keys can be of either analog or digital encoding schemes. Coding information may be transmitted by conventional electrical interfaces (connectors) or via short distance radiofrequency (RF) methods. Software keys may be in the form of a bar code or other passive encoding means. Coding information may be transmitted electrically, optically or by various means known to those skilled in the art.

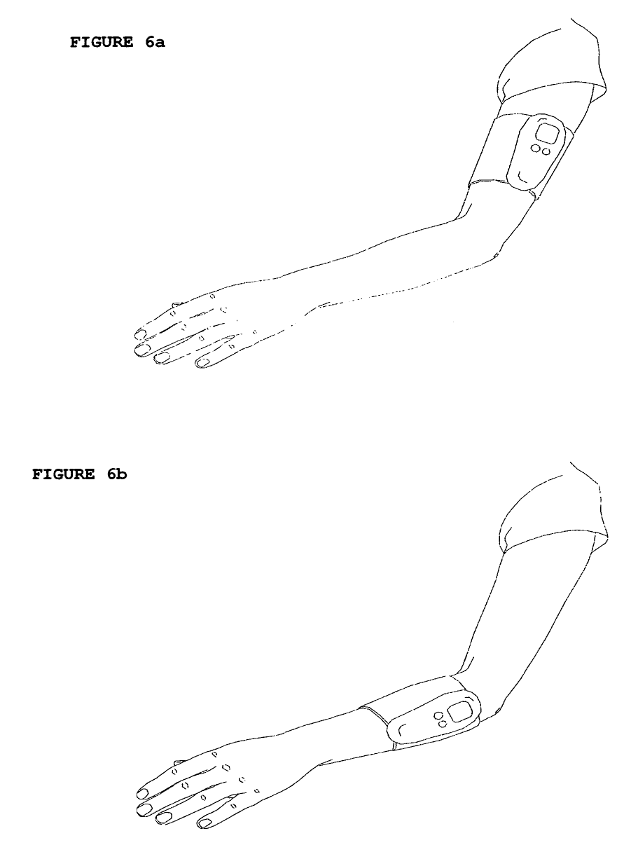

FIG. 5 illustrates a preferred use of a monitor in conjunction with the programmable, automated blood parameter testing device of the present invention. In a preferred embodiment, the automated blood testing device is connected, either via wired links or wireless links, to a portable, optionally hand-held, monitor. Referring to FIG. 5, monitor 500 may comprise a computing automated system such as, but not limited to, a personal digital assistant (PDA), electronic notebook, pager, watch, cellular telephone and electronic organizer. Signals representing blood parameter data obtained from the patient are presented to the monitor which includes both a display 510 and human interface means 520. Using interface means 520 a user may program the device for automatic testing of blood at specified time intervals. The monitor is also provided with a memory 530 to facilitate data archiving and retrieval as may be required.

Optionally, various parameter data from the automated blood testing system may be correlated and analyzed in order to indicate the overall patient condition and/or to indicate critical conditions that require attention. In one embodiment, the control unit of the automatic blood parameter testing device performs this data analysis and/or data correlation. In another embodiment, monitor 500 is equipped with software program 540 for data analysis and correlation. Additionally, software program 540 also supports calculation of trends using look-up tables and algorithms based on measurement history. The results of data analysis and interpretation performed upon the stored patient data by the monitor may optionally be displayed in the form of a paper report generated through a printer (not shown) associated with the monitor 500, besides being displayed on the monitor screen 510.

Software 540 uses a blend of symbolic and numerical methods to analyze the data, detect clinical implications contained in the data and present the pertinent information in the form of a graphics-based data interpretation report. The symbolic methods used by the software encode the logical methodology used by expert diabetologists as they examine patient logs for clinically significant findings, while the numeric or statistical methods test the patient data for evidence to support a hypothesis posited by the symbolic methods which may be of assistance to a reviewing physician.

Optionally, the processed data may be transmitted from the monitor to a central monitoring station when the automatic blood parameter testing device is used in a hospital environment. The central monitoring station maintains a record of all physiological parameters measured over a period of time from different patients. Thus, a plurality of monitors can communicate with the central monitoring station to supply data from various automated blood parameter testing apparatuses.

FIGS. 6a and 6b are schematic diagrams of embodiments of wearable, programmable, automated blood parameter testing apparatus of the present invention. As shown in FIGS. 6a and 6b, in the wearable embodiments of the device of the present invention, the automated blood testing device is housed in a wearable cuff. The wearable cuff may be placed on any suitable location of the body, as in not limited to the following examples. Referring to FIG. 6a, the wearable cuff is placed on the patient's forearm. Referring to FIG. 6b, the wearable cuff is placed on the patient's upper arm. The wearable cuff is preferably secured with an arm band or other suitable attachment mechanism. Other sites, for example the finger, abdomen and leg, are also appropriate for measurement.

In one embodiment the arm band is an inflatable cuff or bladder such as that used with conventional blood pressure measuring automated systems.

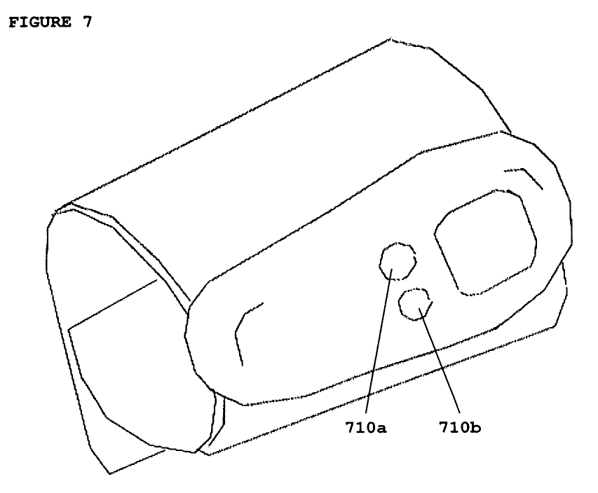

FIG. 7 is a schematic diagram of one embodiment of the automated blood parameter testing apparatus of the present invention, incorporating a pressure cuff. Referring now to FIG. 7, the wearable, programmable blood testing device is operated using control buttons 710a and 710b provided on it. Now referring back to FIG. 2, and also referring to FIG. 7, the operational steps of an integrated pressure cuff and programmable blood testing device are described. When start button (such as 710a) is depressed, the pressure cuff begins to inflate. Simultaneously, automated launching mechanism 230 is actuated, advancing lancet 220, causing lancet 220 to pierce the skin, and retracting lancet 220 after piercing the skin. The inflated pressure cuff facilitates squeezing the blood from the wound in the skin. The blood sample is then collected in reservoir 240, where it was transported via narrow channel 235. The blood sample is then transported to blood analyte measuring element 250.

In a preferred embodiment, the inflatable cuff mechanism is employed for non-invasive measurement of blood pressure. The inflatable cuff acts to occlude peripheral blood flow in the artery. This technique of blood pressure measurement is well known in the art, as will not be described in detail herein.

The above examples are merely illustrative of the many applications of the system of present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention is not to be limited to the details given herein, but may be modified within the scope of the appended claims.

We claim:

1. An automated blood testing device comprising:
a sampling and measurement unit for obtaining a blood sample and measuring blood analytes and blood parameters in said sample, wherein the sampling and measurement unit further comprises a lancet, a lancet launching mechanism, and a blood analyte measuring element; and
a control unit for controlling the periodic sampling of blood and measurement of blood analytes and blood parameters, wherein the control unit is programmable to initiate blood sampling for measurement of blood analytes at pre-determined time intervals wherein said control unit and sampling and measurement unit are positioned in a wearable cuff.

2. The automated device of claim 1 wherein the control unit is a microprocessor or state machine.

3. The automated device of claim 1 wherein the device is capable of being worn on a patient's arm.

4. The automated device of claim 3 wherein the wearable device further comprises an inflatable cuff.

5. The automated device of claim 4 wherein the inflatable cuff is used for obtaining a blood sample via applying pressure.

6. The automated device of claim 4 wherein the inflatable cuff is used for non-invasive measurement of blood pressure.

7. The automated device of claim 4 wherein the inflatable cuff is used for measurement of blood pressure via a capillary parameter.

8. The automated device of claim 1 wherein the lancet withdraws blood from the same point for each sample.

9. The automated device of claim 1 wherein the lancet withdraws blood from a different point for each sample.

10. The automated device of claim 1 wherein the lancet is a single-use lancet.

11. The automated device of claim 1 wherein blood contacting elements are disposable.

12. The automated device of claim 11 wherein blood contacting elements are contained in a disposable cartridge or cassette.

13. The automated device of claim 1 wherein the sampling and measurement unit further comprises at least one measurement element for measuring at least one blood parameter.

14. The automated device of claim 13 wherein the measurement element is a glucose oxidase test strip.

15. The automated device of claim 13 wherein the measurement element is a sensor.

16. The automated device of claim 15 wherein the sensor is contained in a sensor cassette.

17. The automated device of claim 16 wherein the sensor cassette is disposable.

18. The automated device of claim 17 wherein the cassette is coded or keyed to insure proper operation.

19. The automated device of claim 17 wherein the sensor cassette comprises at least one pre-calibrated single use sensor.

20. The automated device of claim 17 wherein the sensor cassette comprises a plurality of sensors arranged in a multiple layer tape structure.

21. The automated device of claim 19 wherein each single-use sensor is advanced sequentially and positioned for direct contact with a blood sample through an advancement means.

22. The automated device of claim 17 wherein the sensor cassette includes a plurality of sensor cassettes, each comprising a different type of sensor.

23. The automated device of claim 15 wherein the sensor is an electrochemical sensor capable of detecting the presence of and enabling the measurement of the level of an analyte in a blood sample via electrochemical oxidation and reduction reactions at the sensor.

24. The automated device of claim 15 wherein the sensor is an optochemical sensor capable of detecting the presence of and enabling the measurement of the level of an analyte in a blood or plasma.

25. The automated device of claim 15 wherein the sensor determines the oxygenation level of the blood and uses the oxygenation level to calibrate the glucose calculation.

26. The automated device of claim 15 wherein the sensor determines the hemoglobin concentration and/or hematocrit of the blood and calibrates the glucose calculation.

27. A method for automatically measuring blood analytes and blood parameters, the method comprising:
programming a control unit to obtain a blood sample from a blood sampling and measurement unit at predetermined time intervals wherein said control unit and blood sampling and measurement unit are positioned in a wearable cuff;
initiating a blood sample from the blood sampling and measurement unit at said predetermined time interval;
launching a lancet via an automated launching mechanism located in said blood sampling and measurement unit when said blood sample is initiated;
allowing said lancet to pierce the skin;
retracting said lancet after said blood sample is obtained; and
measuring analytes and parameters of said blood sample using an analyte measuring element in said blood sampling and measurement unit.
